(19) United States
(12) Patent Application Publication
(10) Pub. No.: US 2010/0152838 A1
(43) Pub. Date: Jun. 17, 2010

(54) Implantable Valvular Prosthesis

(76) Inventors: Wei-Chang Kang, Tainan City (TW); Yu-Li Kang, Tainan City (TW)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/335,308

(22) Filed: Dec. 15, 2008

(51) Int. Cl. A61F 2/06; A61F 2/24

(52) U.S. Cl. 623/1.24; 623/2.1; 623/1.36

(57) ABSTRACT

An implantable valvular prosthesis is adapted for replacement of an anatomic valve that controls passage of blood flowing from an atrium to a ventricle in a patient's heart. The implantable valvular prosthesis includes a tubular stent body, at least one valve flap member, and a graft member.

The tubular stent body includes a tubular trailing region and a tubular leading region. The valve flap member is disposed to associate with the tubular stent body. The graft member covers an abluminal surface of the tubular stent body.

### BACKGROUND OF THE INVENTION

1. Field of the Invention

This invention relates to a prosthesis, more particularly to an implantable valvular prosthesis.

2. Description of the Related Art

A conventional valvular prosthesis is suitable for replacing an anatomic valve that controls passage of blood flowing from an atrium to a ventricle in a patient's heart. The atrium has a first luminal wall having a tubular valve seat region which borders a second luminal wall defining the ventricle, and to which a base portion of the anatomic valve is connected along an inner periphery of the tubular valve seat region.

Referring to FIGS. 1 and 2, the conventional valvular prosthesis 10 for replacing a mitral valve 14 is tubular and elastic, and includes a top anchor section 102, a bottom anchor section 103, an intermediate section that interconnects the top and bottom anchor sections 102, 103, and valve flaps. The valvular prosthesis 10 is able to replace a tricuspid valve 15 as well. The valvular prosthesis 10 is percutaneously delivered to a tubular valve seat region that pertains to a first luminal wall of a left atrium 12 by virtue of cardiac catheterization. When the valvular prosthesis 10 is deployed at the tubular valve seat region, the bottom anchor section 103 is expanded to exclude the mitral valve 14 from a closed position to an open position. The intermediate section is expanded to contact the tubular valve seat region. The top anchor section 102 is expanded to contact the first luminal wall of the left atrium 12.

Since the intermediate section has a shape that is not conformable to a shape of the tubular valve seat region, the intermediate section is unable to entirely contact the tubular valve seat region. Therefore, a space 130 between the intermediate section and the tubular valve seat region is formed. When the valve flaps are biased to a closed position, blood in a left ventricle 11 is able to regurgitate into the left atrium 12 through the space 130.

The mitral valve 14 is closely adjacent to an aortic valve 17 and has two cusp portions. When the bottom anchor section 103 is expanded to exclude the mitral valve 14, one of the cusp portions of the mitral valve 14 is pushed toward the aortic valve 17. Furthermore, the cusp portions of the mitral valve 14 are unable to be biased to a closed position during systole. Thus, blood flow from the left ventricle 11 to a sinus of Valsalva 16 is impeded by one of the cusp portions of the mitral valve 14.

### SUMMARY OF THE INVENTION

Therefore, the object of the present invention is to provide an implantable valvular prosthesis that can overcome the aforesaid drawbacks of the prior art.

According to this invention, an implantable valvular prosthesis is adapted for replacement of an anatomic valve which controls passage of blood flowing from an atrium to a ventricle in a patient's heart. The atrium has a first luminal wall having a tubular valve seat region which borders a second luminal wall defining the ventricle, and to which a base portion of the anatomic valve is connected along an inner periphery of the tubular valve seat region. The implantable valvular prosthesis includes a tubular stent body, at least one valve flap member, and a graft member.

The tubular stent body defines a central longitudinal axis, and is made from a material expand able at a site of implantation. The tubular stent body has an abluminal surface to surround the central longitudinal axis, and a luminal surface that is opposite to the abluminal surface in an axial direction and that defines a central tubular opening along the central longitudinal axis. The tubular stent body includes a tubular trailing region and a tubular leading region.

The tubular trailing region, when expanded, is disposed to extend into the atrium so as to permit the blood in the atrium to flow therethrough.

The tubular leading region is opposite to the tubular trailing region along the central longitudinal axis, and is disposed downstream of the tubular trailing region in terms of blood flow under diastole pressure. The tubular leading region is configured such that once the tubular leading region is brought from the atrium to the tubular valve seat region to be expanded in-situ, the tubular leading region is anchored to the tubular valve seat region so as to be in intimate contact therewith along the inner periphery of the tubular valve seat region when the tubular valve seat region is distended as a result of the diastole pressure, and such that the tubular leading region is kept to steer clear of the movement of a cusp portion of the anatomic valve toward a closed position during systole.

The valve flap member is disposed to associate with the tubular stent body, extends from the luminal surface of the tubular stent body towards the central longitudinal axis, and is biased under zero pressure differential to a closed position where a passage of the blood is interrupted from flowing through the central tubular opening.

The graft member covers the abluminal surface of the tubular stent body so as to ensure that the central tubular opening is a sole available route for the blood flowing from the atrium to the ventricle.

### BRIEF DESCRIPTION OF THE DRAWINGS

Other features and advantages of the present invention will become apparent in the following detailed description of the preferred embodiments of this invention, with reference to the accompanying drawings, in which:

FIG. 1 is a schematic partly cross-sectional view to illustrate a conventional valvular prosthesis implanted in a position of a tubular valve seat region that pertains to a first luminal wall of a left atrium;

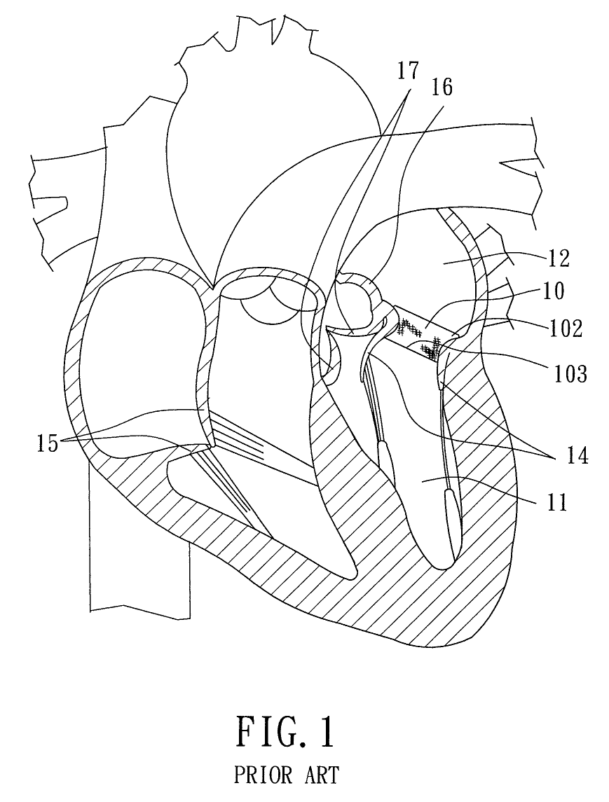

FIG. 2 is a schematic partly sectional view to illustrate the conventional valvular prosthesis implanted in the position of the tubular valve seat region that pertains to the first luminal wall of the left atrium;

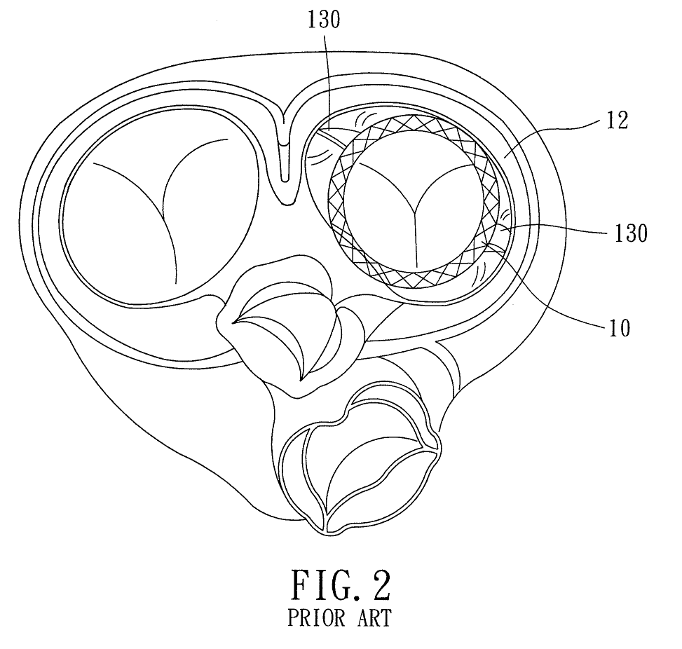

FIG. 3 is a schematic view of the first preferred embodiment of an implantable valvular prosthesis according to this invention;

FIG. 4 is a schematic view to illustrate a tubular stent body of the implantable valvular prosthesis shown in FIG. 3;

FIG. 5 is a schematic view to illustrate three valve flap members disposed to associate with the tubular stent body shown in FIG. 4;

FIG. 6 is a schematic view illustrating how the implantable valvular prosthesis in FIG. 3 is placed in a catheter;

FIG. 7 is a schematic partly cross-sectional view illustrating how the implantable valvular prosthesis in FIG. 3 is implanted in a position of a tubular valve seat region that pertains to a first luminal wall of a left atrium during diastole;

FIG. 8 is a schematic partly cross-sectional view illustrating how the implantable valvular prosthesis in FIG. 3 is implanted in the position of the tubular valve seat region that pertains to the first luminal wall of the left atrium during systole;

FIG. 9 is a schematic view illustrating how the implantable valvular prosthesis in FIG. 3 is implanted in the position of the tubular valve seat region that pertains to the first luminal wall of the left atrium;

FIG. 10 is a schematic view of the second preferred embodiment of the implantable valvular prosthesis according to this invention;

FIG. 11 is a schematic partly cross-sectional view illustrating how the implantable valvular prosthesis in FIG. 10 is implanted in a position of a tubular valve seat region that pertains to a first luminal wall of a right atrium; and

FIG. 12 is a schematic view illustrating how the implantable valvular prosthesis in FIG. 10 is implanted in the position of the tubular valve seat region that pertains to the first luminal wall of the right atrium.

### DETAILED DESCRIPTION OF THE PREFERRED EMBODIMENTS

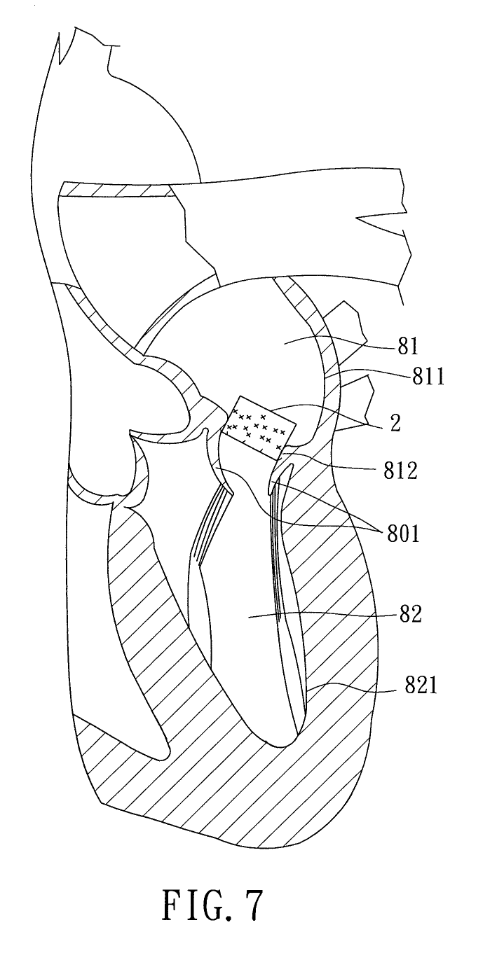

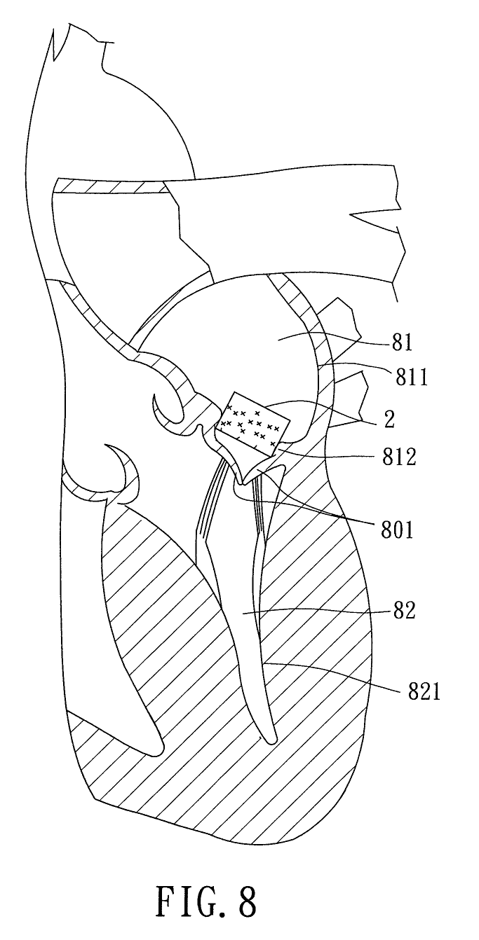

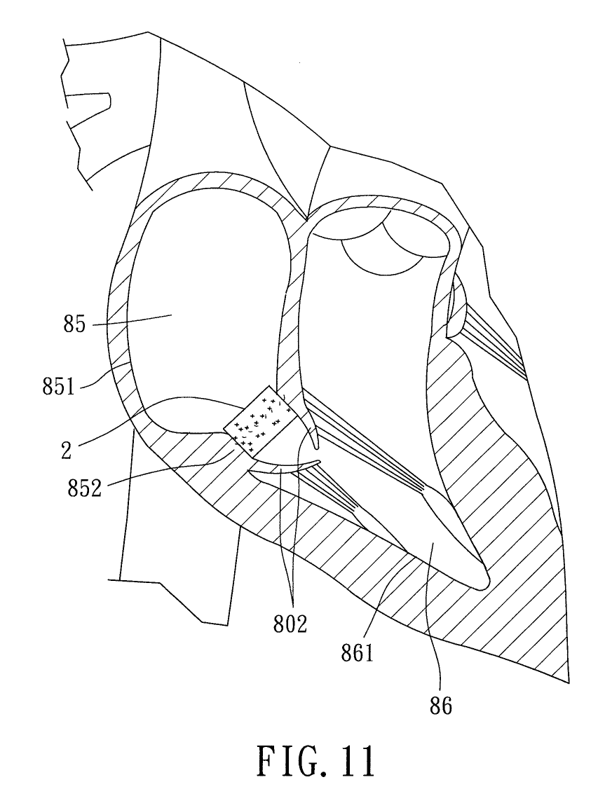

Referring to FIGS. 7 and 8, the first preferred embodiment of an implantable valvular prosthesis 2 according to the present invention is adapted for replacement of a mitral valve 801 which controls passage of blood flowing from a left atrium 81 to a left ventricle 82 in a patient's heart. The left atrium 81 has a first luminal wall 811 having a tubular valve seat region 812 which borders a second luminal wall 821 defining the left ventricle 82, and to which a base portion of the mitral valve 801 is connected along an inner periphery of the tubular valve seat region 812. Preferably, the implantable valvular prosthesis 2 in the first preferred embodiment is implanted in a position of the tubular valve seat region 812 that pertains to the first luminal wall 811 of the left atrium 81. It should be noted that the implantable valvular prosthesis 2 in the first preferred embodiment is able to be implanted in a position of a tubular valve seat region 852 (shown in FIG. 11) that pertains to a first luminal wall 851 (shown in FIG. 11) of a right atrium 85 (shown in FIG. 11) as well.

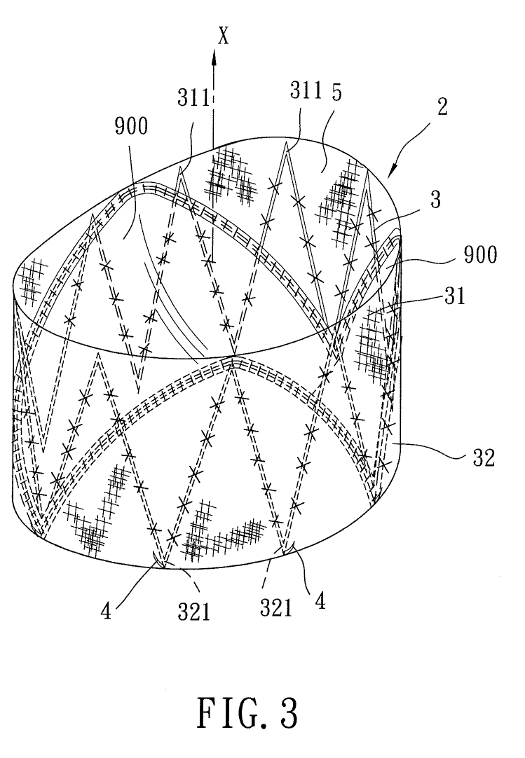

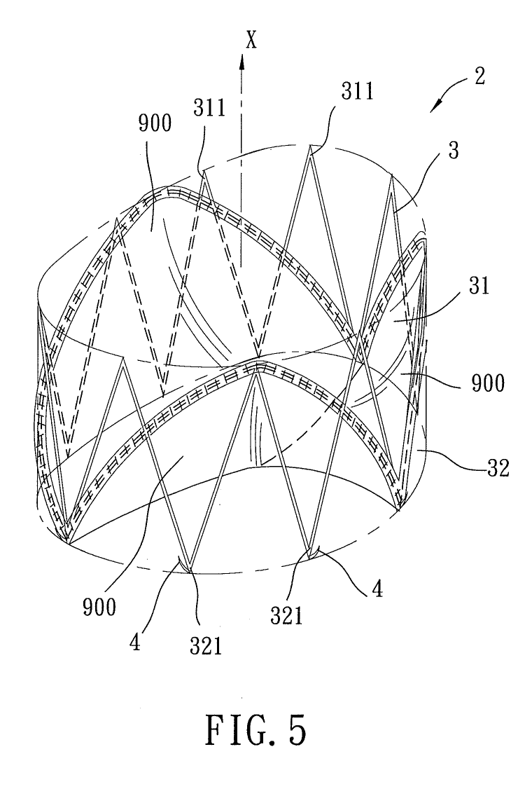

Referring to FIGS. 3, 4, and 5, the implantable valvular prosthesis 2 includes a tubular stent body 3, three valve flap members 900, and a graft member 5. It is noted that an amount of the valve flap members 900 can be adjusted to meet requirements.

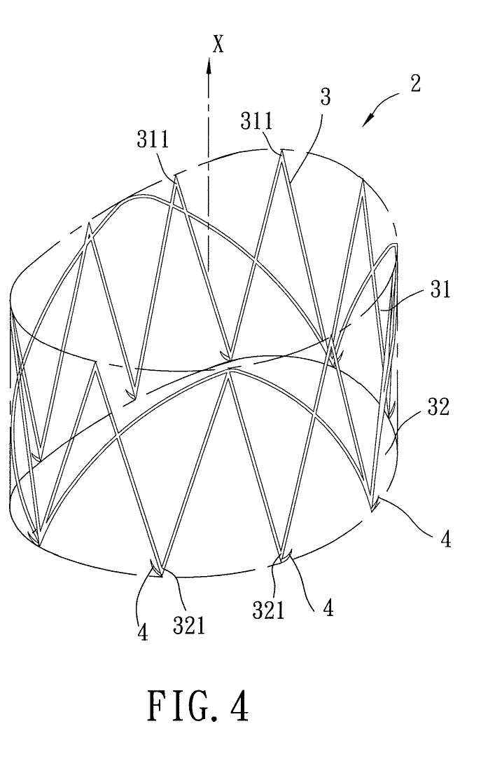

The tubular stent body 3 defines a central longitudinal axis (X), and is made from a material expand able at a site of implantation. In this embodiment, the tubular stent body 3 is made from a plurality of metal struts that are made from a shape memory material, and that intersect with each other (see FIG. 4). The tubular stent body 3 has an abluminal surface to surround the central longitudinal axis (X), and a luminal surface that is opposite to the abluminal surface in an axial direction and that defines a central tubular opening along the central longitudinal axis (X). The tubular stent body 3 includes a tubular trailing region 31 and a tubular leading region 32.

The tubular trailing region 31 has a plurality of apexes 311 that are formed by the intersections of the metal struts.

The tubular leading region 32 has a plurality of apexes 321 that are formed by the intersections of the metal struts, is opposite to the tubular trailing region 31 along the central longitudinal axis (X), and is disposed downstream of the tubular trailing region 31 in terms of blood flow under diastole pressure.

The valve flap members 900 are disposed to associate with the tubular stent body 3, extend from the luminal surface of the tubular stent body 3 towards the central longitudinal axis (X), and are biased under zero pressure differential to a closed position where a passage of the blood is interrupted from flowing through the central tubular opening. In this embodiment, the valve flap members 900 are sewn to the tubular stent body 3.

The graft member 5 covers the abluminal surface of the tubular stent body 3 so as to ensure that the central tubular opening is a sole available route for the blood flowing from the atrium (the left atrium 81 in this embodiment) to the ventricle (the left ventricle 82 in this embodiment).

The implantable valvular prosthesis 2 further includes a plurality of anchoring needles 4 which are configured when the tubular stent body 3 is expanded, such that the anchoring needles 4 are brought to engage the tubular valve seat region (the tubular valve seat region 812 that pertains to the first luminal wall 811 of the left atrium 81 in this embodiment) and to anchor thereat. In this embodiment, the anchoring needles 4 extend upwardly and obliquely from the apexes 321 of the tubular leading region 32.

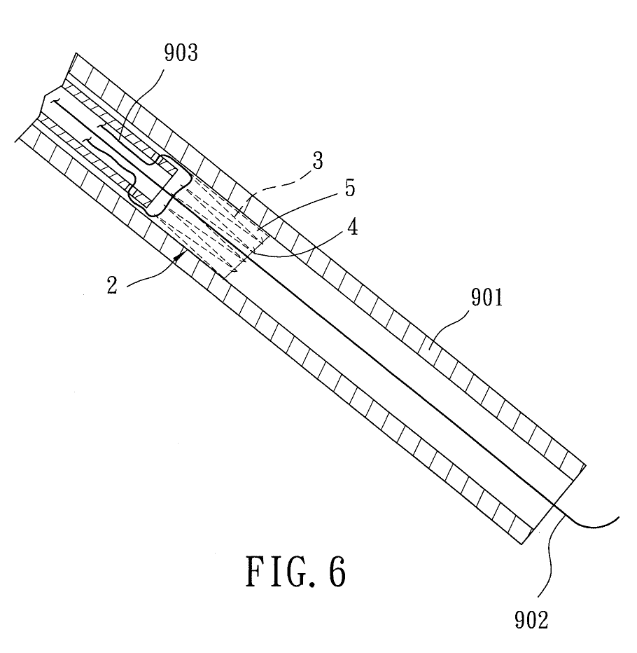

Referring to FIGS. 3 and 6, a guide wire 902 extends longitudinally through the tubular stent body 3. A pulling string 903 extends through the tubular trailing region 31 in order to drag the apexes 311 of the tubular trailing region 31. By virtue of the guide wire 902 and the pulling string 903, the tubular stent body 3 can be stretched in a direction along the central longitudinal axis (X) and contracted inwardly toward the central longitudinal axis (X). Therefore, the tubular stent body 3 can be placed into a catheter 901. Since the feature of the invention does not reside in a method of placing the tubular stent body 3 into the catheter 901, further details of the same are omitted herein for the sake of brevity.

Referring to FIGS. 3, 6, and 7, during cardiac catheterization, after one end of the catheter 901 is delivered to the tubular valve seat region 812 that pertains to the first luminal wall 811 of the left atrium 81, the tubular leading region 32 and the tubular trailing region 31 are withdrawn from the catheter 901 in sequence. The tubular leading region 32 withdrawn from the catheter 901 is expanded and anchored to the tubular valve seat region 812 so as to be in intimate contact therewith along the inner periphery of the tubular valve seat region 812. The anchoring needles 4 on the apexes 321 of the tubular leading region 32 are brought to pierce upwardly and obliquely into the tubular valve seat region 812. Afterward, the tubular trailing region 31 withdrawn from the catheter 901 is expanded and disposed to extend into the left atrium 81 so as to permit blood in the left atrium 81 to flow therethrough.

Referring to FIGS. 3, 7, and 8, the tubular leading region 32 is configured such that the tubular leading region 32 is kept to steer clear of the movement of two cusp portions of the mitral valve 801 toward a closed position during systole. Thus, blood flow from the left ventricle 82 to a sinus of Valsalva is not impeded by the mitral valve 801. In addition, the mitral valve 801 is capable of assisting the valve flap members 900 of the implantable valvular prosthesis 2 in controlling the passage of the blood flowing from the left atrium 81 to the left ventricle 82.

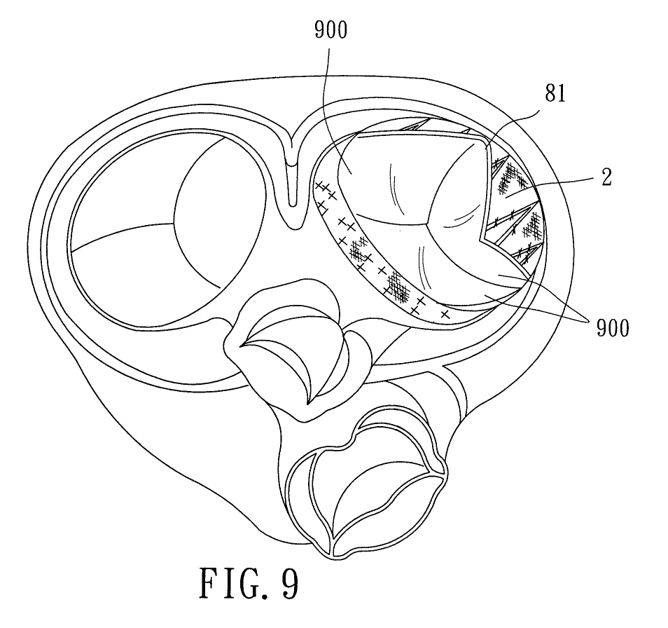

Referring to FIGS. 3, 7, and 9, the tubular leading region 32 is capable of entirely and intimately contacting the tubular valve seat region 812 along the inner periphery of the tubular valve seat region 812 because a shape of the tubular leading region 32 is conformable to a shape of the tubular valve seat region 812 that pertains to the first luminal wall 811 of the left atrium 81. Furthermore, the tubular leading region 32 is configured such that the tubular leading region 32 is brought to entirely and intimately contact the tubular valve seat region 812 along the inner periphery of the tubular valve seat region 812 when the tubular valve seat region 812 is distended as a result of diastole pressure. Consequently, during the systole or the diastole, there is little or no space formed between the tubular leading region 32 and the tubular valve seat region 812. When the valve flap members 900 are biased under zero pressure differential to a closed position, blood in the left ventricle 82 is unable to regurgitate into the left atrium 81.

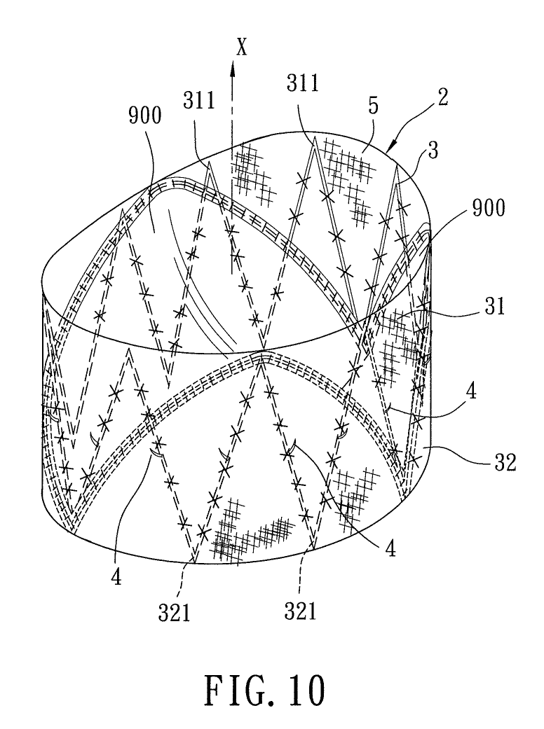

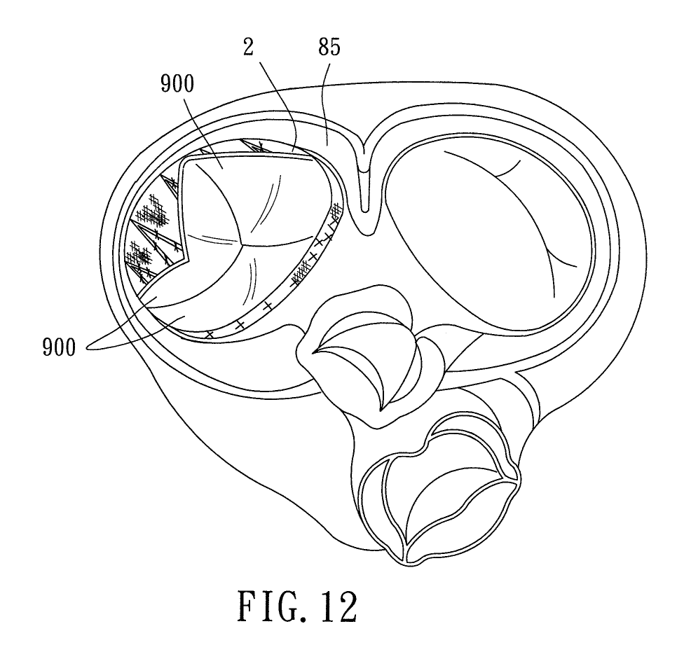

Referring to FIGS. 10, 11, and 12, the second preferred embodiment of the implantable valvular prosthesis 2 according to this invention is illustrated. The structure of this preferred embodiment is similar to the structure of the first preferred embodiment. The difference between this embodiment and the first preferred embodiment resides in that the anchoring needles 4 extend upwardly and obliquely from portions that pertain to the metal struts and that are between the apexes 311, 321 of the tubular trailing region 31 and the tubular leading region 32.

Preferably, the implantable valvular prosthesis 2 in the second preferred embodiment is implanted in the position of the tubular valve seat region 852 that pertains to the first luminal wall 851 of the right atrium 85 for replacing the tricuspid valve 802. It should be noted that the implantable valvular prosthesis 2 in the second preferred embodiment is able to be implanted in the position of the tubular valve seat region 812 (shown in FIG. 7) that pertains to the first luminal wall 811 (shown in FIG. 7) of the left atrium 81 (shown in FIG. 7) as well.

The right atrium 85 has the first luminal wall 851 having the tubular valve seat region 852 which borders a second luminal wall 861 defining a right ventricle 86, and to which a base portion of the tricuspid valve 802 is connected along an inner periphery of the tubular valve seat region 852.

Since cardiac catheterization that is used in order to deliver the implantable valvular prosthesis 2 to the tubular valve seat region 852 pertaining to the first luminal wall 851 of the right atrium 85 is similar to the cardiac catheterization that is used for the first preferred embodiment, further details of the same are omitted herein for the sake of brevity.

The tubular trailing region 31 is expanded and disposed to extend into the right atrium 85 so as to permit blood in the right atrium 85 to flow therethrough.

The tubular leading region 32 is configured such that the tubular leading region 32 is kept to steer clear of the movement of three cusp portions of the tricuspid valve 802 toward a closed position during systole. Thus, blood flow from the right ventricle 86 to a sinus of Valsalva is not impeded by the tricuspid valve 802.

The tubular leading region 32 is capable of entirely and intimately contacting the tubular valve seat region 852 along the inner periphery of the tubular valve seat region 852 because a shape of the tubular leading region 32 is conformable to a shape of the tubular valve seat region 852 that pertains to the first luminal wall 851 of the right atrium 85. Furthermore, the tubular leading region 32 is configured such that the tubular leading region 32 is brought to entirely and intimately contact the tubular valve seat region 852 along the inner periphery of the tubular valve seat region 852 when the tubular valve seat region 852 is distended as a result of diastole pressure. Consequently, during the systole or the diastole, there is little or no space formed between the tubular leading region 32 and the tubular valve seat region 852. When the valve flap members 900 are biased under zero pressure differential to a closed position, blood in the right ventricle 86 is unable to regurgitate into the right atrium 85.

While the present invention has been described in connection with what are considered the most practical and preferred embodiments, it is understood that this invention is not limited to the disclosed embodiments but is intended to cover various arrangements included within the spirit and scope of the broadest interpretation and equivalent arrangements.

What is claimed is:

1. An implantable valvular prosthesis adapted for replacement of an anatomic valve which controls passage of blood flowing from an atrium to a ventricle in a patient's heart, the atrium having a first luminal wall that has a tubular valve seat region which borders a second luminal wall defining the ventricle, and to which a base portion of the anatomic valve is connected along an inner periphery of the tubular valve seat region, said implantable valvular prosthesis comprising:
a tubular stent body defining a central longitudinal axis, and made from a material expand able at a site of implantation, said tubular stent body having an abluminal surface to surround the central longitudinal axis, and a luminal surface that is opposite to said abluminal surface in an axial direction and that defines a central tubular opening along the central longitudinal axis, said tubular stent body including
a tubular trailing region which, when expanded, is disposed to extend into the atrium so as to permit the blood in the atrium to flow therethrough; and
a tubular leading region which is opposite to said tubular trailing region along the central longitudinal axis, and which is disposed downstream of said tubular trailing region in terms of blood flow under diastole pressure, said tubular leading region being configured such that once said tubular leading region is brought from the atrium to the tubular valve seat region to be expanded in-situ, said tubular leading region is anchored to the tubular valve seat region so as to be in intimate contact therewith along the inner periphery of the tubular valve seat region when the tubular valve seat region is distended as a result of the diastole pressure, and such that said tubular leading region is kept to steer clear of the movement of a cusp portion of the anatomic valve toward a closed position during systole;
at least one valve flap member disposed to associate with said tubular stent body, extending from said luminal surface of said tubular stent body toward the central longitudinal axis, and biased under zero pressure differential to a closed position where a passage of the blood is interrupted from flowing through said central tubular opening; and
a graft member covering said abluminal surface of said tubular stent body so as to ensure that said central tubular opening is a sole available route for the blood flowing from the atrium to the ventricle.

2. The implantable valvular prosthesis as claimed in claim 1, further comprising a plurality of anchoring needles which are configured when said tubular stent body is expanded, such that said anchoring needles are brought to engage the tubular valve seat region and to anchor thereat.
